(19) United States
(12) Patent Application Publication
(10) Pub. No.: US 2014/0288878 A1
(43) Pub. Date: Sep. 25, 2014

(54) IDENTIFICATION OF MOTION CHARACTERISTICS TO DETERMINE ACTIVITY

(71) Applicant: Thomas Alan Donaldson, Nailsworth (GB)

(72) Inventor: Thomas Alan Donaldson, Nailsworth (GB)

(73) Assignee: AliphCom, San Francisco, CA (US)

(21) Appl. No.: 14/207,263

(22) Filed: Mar. 12, 2014

Related U.S. Application Data
(60) Provisional application No. 61/802,303, filed on Mar. 15, 2013.

(51) Int. Cl. A61B 5/11; G01P 13/00

(52) U.S. Cl. CPC A61B 5/1123; G01P 13/00
USPC 702/141

(57) ABSTRACT

Embodiments of the relate generally to electrical and electronic hardware, computer software, wired and wireless network communications, and wearable computing devices for facilitating health and wellness-related information. More specifically, disclosed are systems, methods, devices, computer readable medium, and apparatuses configured to determine activity and activity types, including gestures, from sensed motion signals using, for example, a wearable device (or carried device) and one or more motion sensors. In some embodiments, a method can include receiving data representing a motion sensor signal from a motion sensor disposed in a wearable device, and generating intermediate motion signals from the motion sensor signal. The method also can include identifying characteristics of motion based on the intermediate motion signals to form motion characteristics data, and determining an activity based the motion characteristics data.

### CROSS-REFERENCE TO RELATED APPLICATIONS

This application is a U.S. non-provisional patent application that claims the benefit of U.S. Provisional Patent Application No. 61/802,303, filed Mar. 15, 2013, and entitled “IDENTIFICATION OF MOTION CHARACTERISTICS TO DETERMINE ACTIVITY,” which is herein incorporated by reference for all purposes.

### FIELD

Embodiments of the invention relate generally to electrical and electronic hardware, computer software, wired and wireless network communications, and wearable computing devices for facilitating health and wellness-related information. More specifically, disclosed are systems, methods, devices, computer readable medium, and apparatuses configured to determine activity and activity types, including gestures, from sensed motion signals using, for example, a wearable device (or carried device) and one or more motion sensors.

### BACKGROUND

While functional, conventional devices and techniques to gather activity information based on sensed motion, such as activity information for identifying walking or running as an activity, are not well-suited to accurately and precisely analyze motion and address the inaccuracies that are common in traditional approaches to using motion sensors, such as accelerometers.

For example, accelerometers typically have very significant offsets, such as 60 mg, or greater, and have sensitivity errors of up to 2-3%. Conventional accelerators also experience cross-coupling between axes of, for example, 1-2%. These wide variances can affect many algorithms and influence the results deleteriously. This can throw off estimates of orientation, etc. Further, calibration of accelerometers typically requires a device to be moved through a known path, typically at manufacturing, and this can be time consuming and expensive. Moreover, calibration values also change over time as drift can occur.

Some conventional motion sensing and applications are susceptible to relatively large amounts of power consumption, which scales with sample rate. Further, certain activities, like running, typically have energy disposed at higher frequencies than other activities, such as sleeping. To capture running data, sampling rates are typically set higher (i.e., oversampling) than may be required, for example, during low-level activities, leading to undesired power consumption.

Further, conventional approaches normally operate on raw motion (i.e., accelerometer) signals, which usually inject uncertainty and inaccuracies in classifying motion with a type of activity. Thus, amounts of activity are typically determined with wide tolerances, which, sometimes, may be of little value to a user. Rather than describing amounts of activities, a few approaches rely on tracking “points” as a measure of activity with tenuous relationships to the actual underlying activity.

Common motion analyzation techniques in determining aspects of activities are not well-suited for a variety of applications. For example, some approaches are susceptible to spectral distortion as they operate at a fraction of the sample rate. Other approaches have poor temporal resolution at high frequencies, and can have excessive temporal resolution at low frequencies. They can also be computationally difficult for some processors to provide such analysis as they may not be specifically designed for the purpose.

Thus, what is needed is a solution for capturing motion for determining activities, such as motion associated with wearable devices, without the limitations of conventional techniques.

### BRIEF DESCRIPTION OF THE DRAWINGS

Various embodiments or examples (“examples”) of the invention are disclosed in the following detailed description and the accompanying drawings:

FIG. 1 illustrates an exemplary device for determining motion and activities that is disposed in a wearable device, according to some embodiments;

FIG. 2 is a diagram depicting a signal preprocessor, according to some embodiments;

FIG. 3 is an example flow diagram for calibrating a motion sensor in-line, according to some embodiments;

FIG. 4 illustrates a calibrated motion signal, according to at least one example;

FIG. 5 is an example flow diagram for dynamically controlling a sample rate, according to some embodiments;

FIG. 6 is an example of an intermediate motion signal generator, according to some embodiments;

FIG. 7 is a diagram depicting an estimated orientation derived from an intermediate motion signal generator, according to some embodiments;

FIG. 8 is a diagram depicting a motion characteristic identifier, according to some examples;

FIG. 9 is an example of a dynamic emphasizer, according to some embodiments;

FIG. 10 depicts extracted features according to some embodiments;

FIG. 11 depicts an activity classifier, according to some embodiments; and

FIG. 12 illustrates an exemplary computing platform disposed in a wearable device or otherwise implements at least some of the various components in accordance with various embodiments.

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

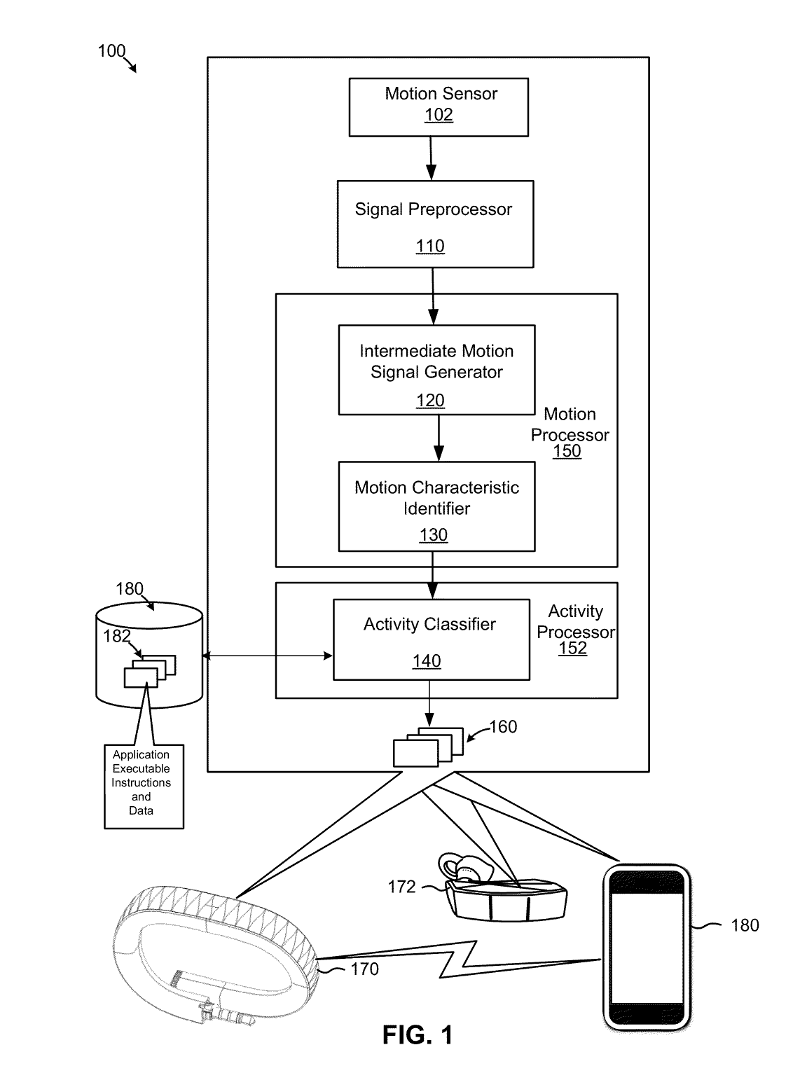

FIG. 1 illustrates an exemplary device for determining motion and activities that is disposed in a wearable device, according to some embodiments. Diagram 100 depicts a device 101 including a motion sensor 102, such as an accelerometer, or any other type of sensor, a signal preprocessor 110, an intermediate motion signal generator 120, a motion characteristic identifier 130, and an activity classifier 140, which is configured to generate data 160 describing an activity one or more characteristics of that activity as well as parameters thereof. Device 101 can be disposed in a wearable device 170 including a wearable housing, a headset 172, as a wearable device, in a mobile device 180, or any other device. As shown, motion processor 150 includes intermediate motion signal generator 120 and motion characteristic identifier 130. An activity processor 152 includes activity classifier 140 is coupled to a repository 180 that includes application data and hence executable instructions 182. In one embodiment, motion processor 150 is a digital signal processor and activity processor 152 is a microcontroller but either of which can be any processor.

In some embodiments, wearable device 170 can be in communication (e.g., wired or wirelessly) with a mobile device 180, such as a mobile phone or computing device. In some cases, mobile device 180, or any networked computing device (not shown) in communication with wearable device 170, 172 or mobile device 180, can provide at least some of the structures and/or functions of any of the features described herein. As depicted in FIG. 1 and subsequent figures, the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in FIG. 1 (or any subsequent figure) can represent one or more algorithms. Or, at least one of the elements can represent a portion of logic including a portion of hardware configured to provide constituent structures and/or functionalities.

For example, a signal preprocessor 110, an intermediate motion signal generator 120, a motion characteristic identifier 130, and an activity classifier 140, can be implemented in one or more computing devices (i.e., any mobile computing device, such as a wearable device or mobile phone, whether worn or carried) that include one or more processors configured to execute one or more algorithms in memory. Thus, at least some of the elements in FIG. 1 (or any subsequent figure) can represent one or more algorithms. Or, at least one of the elements can represent a portion of logic including a portion of hardware configured to provide constituent structures and/or functionalities. These can be varied and are not limited to the examples or descriptions provided.

As hardware and/or firmware, the above-described structures and techniques can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit. For example, a signal preprocessor 110, an intermediate motion signal generator 120, a motion characteristic identifier 130, and an activity classifier 140, can be implemented in one or more computing devices that include one or more circuits. Thus, at least one of the elements in FIG. 1 (or any subsequent figure) can represent one or more components of hardware. Or, at least one of the elements can represent a portion of logic including a portion of circuit configured to provide constituent structures and/or functionalities.

According to some embodiments, the term “circuit” can refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit can include a system of electronic components and logic components (e.g., logic configured to execute instructions, such that a group of executable instructions of an algorithm, for example, and, thus, is a component of a circuit). According to some embodiments, the term “module” can refer, for example, to an algorithm or a portion thereof, and/or logic implemented in either hardware circuitry or software, or a combination thereof (i.e., a module can be implemented as a circuit). In some embodiments, algorithms and/or the memory in which the algorithms are stored are “components” of a circuit. Thus, the term “circuit” can also refer, for example, to a system of components, including algorithms. These can be varied and are not limited to the examples or descriptions provided.

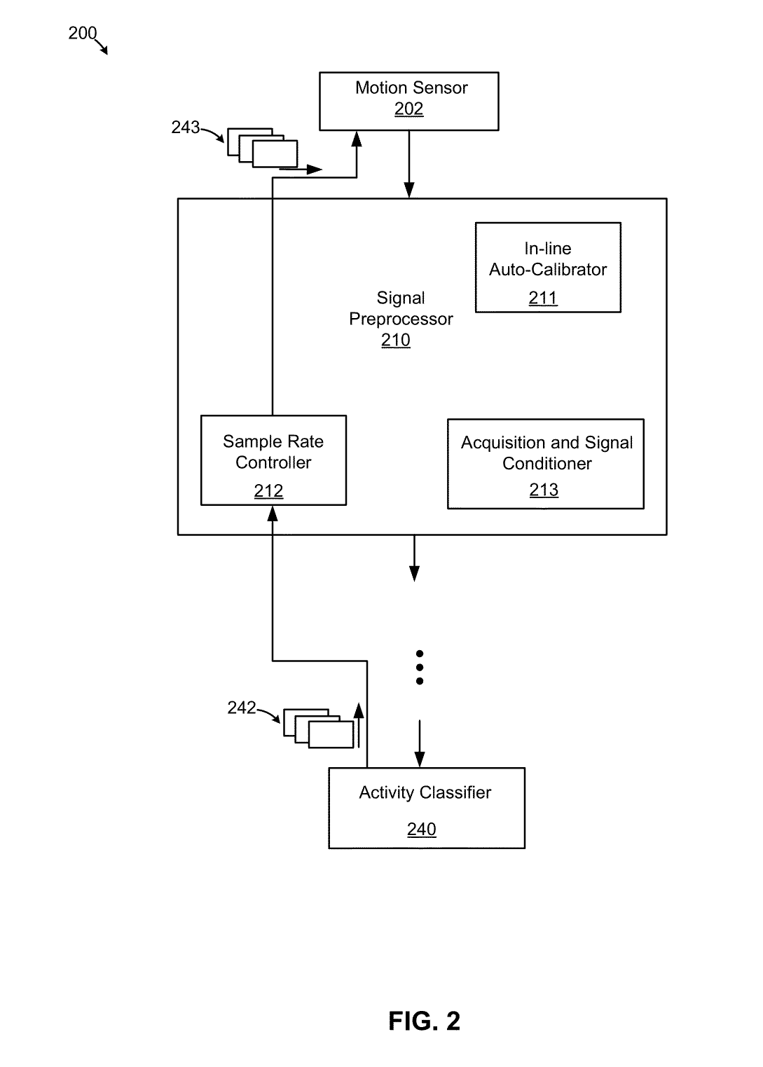

FIG. 2 is a diagram depicting a signal preprocessor, according to some embodiments. Diagram 200 depicts a signal preprocessor 210 configured to receive motion signals from a motion sensor 202. An example of a motion sensor 202, is an accelerometer but can be any other type of sensor that can detect motion including gyroscopes, magnetometers, etc., any of which can be implemented in cooperation with an accelerometer. As shown, preprocessor 210 includes an in-line auto-calibrator 211, an acquisition and signal conditioner 213, and a sample rate controller 212. Signal preprocessor 210 is configured to optimize signal quality while maintaining a minimal cost (i.e., in terms of power consumption, etc.). In particular, signal preprocessor 210 is configured to minimize the sampling of noise and compensate for device-to-device and use-to-use differences while reducing loss of data. For example, signal preprocessor 210 can be configured to reduce clipping due to accelerations that exceed a current range, quantization due to accelerations being lower than the least significant bit (“LSB”) of the current range, and/or signals having energy at a higher frequency than the current Nyquist frequency. Examples of device-to-device and use-to-use differences may arise due to offsets and sensitivity errors in a device, differently sized devices, and different configurations of wearing a wearable device, such as a wristband device, each configuration introducing a different coordinate system for motion determinations.

Acquisition and signal conditioner 213 is configured to compensate for different configurations of a wearable device. There may, for example, be at least four ways of wearing an UP™ band, depending on whether a button is implemented (if at all) on the inner or outer wrist, or whether the button is facing in toward the body or away from a body of a user. Each configuration may give rise to a coordinate rotation applied to movements of the body. As movements of a wearable device can involve movement of the forearm, if, for example, the device is worn at or near a wrist. These movements may include rotation around the elbow, which, in turn, may give rise to a centripetal acceleration (e.g., towards the elbow). In some embodiments, a bias can be determined from a distribution of centripetal accelerations, such as those accelerations associated with a radius of curvature of an order of magnitude of an “elbow-to-wrist” distance. Acquisition and signal conditioner 213, therefore, can use the bias to estimate the configuration (e.g., the manner or orientation in which a wearable device is coupled to a body relative to a portion of a body, such as a limb). A rotation can be determined and then applied to the input stream of motion data, such as an accelerometer stream.

In-line auto-calibrator 211 is configured to recalibrate an accelerometer, continuously while in-situ to reduce time-varying offsets and gain errors. When performing calibration, in-line auto-calibrator 211 is configured to detect whether the accelerometer is still (e.g., in any orientation), and if so, in-line auto-calibrator 211 performs the recalibration. For example, in-line auto-calibrator 211 can be configured to determine the power spectral density (e.g., over 2 to 4 seconds) and subtract a unit of 1 G from a DC component. Further, in-line auto-calibrator 211 can compare the total amount of energy with a noise floor of motion sensor 202. Then, in-line auto-calibrator 211 can estimate the current orientation for the wearable device, and determine a value of an acceleration due to gravity, g, that should be applied to the wearable device for the current orientation. Next, in-line auto-calibrator 211 can subtract the actual acceleration values from the estimated values, to determine an offset as the mean of the differences, and a sensitivity error as, for example, the actual value divided by an estimated value. In-line auto-calibrator 211 can iterate the calibration process to minimize the above-described values.

In some cases, in-line auto-calibrator 211 can detect whether motion sensor 202 is indicating a wearable device is still by determining the power spectral density and subtracting an average value of a DC frequency bin from the value of the DC bin. Then, in-line auto-calibrator 211 can obtain an RMS value of the remaining values for the other frequency bins. The result is compared against a threshold value, which indicates whether the RMS value of the accelerometer noise indicates that the wearable device is still. If still, in-line auto-calibrator 211 can estimate an acceleration due to gravity as being 1 G in the direction of the measured acceleration. Without limitation, an example value of “g” can be determined as being 1 G*normal acceleration. Any residual acceleration ought to be zero that is, a value of the current acceleration subtracted from the estimate of the value of gravity, G, ought to be zero to determine an offset in a gain error. In this case, the offset is determined as being a median error, whereas the gain error is the mean gain. In-line auto-calibrator 211 iterates the calibration process to ensure errors due to rotation of estimated orientation can be reduced or negated.

Sample rate controller 212 is configured to optimize power consumption based on controlling the sample rate at which the motion sensor 202 is sampled. In some embodiments, sample rate controller 212 is configured to receive usage data 242 from an activity classifier 240, whereby the usage data 242 indicates an amount of activity associated with the wearable device. For example, usage data 242 can indicate a high level of activity if the wearable device is experiencing large amounts of motion as a user is running. However, the usage data may indicate a relatively low level of activity if the user is resting or sleeping. Sample rate controller 212 uses this information to determine whether to increase the sample rate to capture sufficient amounts of data during high levels of activity when there is likely relatively large amounts of variation in the motion data, or decrease a sample rate to sufficiently capture motion data to conserve power. Sample rate controller 212 provides control data 243 to motion sensor 202 for purposes of controlling operation of, for example, an accelerometer.

Sample rate controller 212 is configured to monitor the signal spectrum of the accelerometer data stream, and to adjust sample rate accordingly. In at least some examples, sample rate controller 212 is configured to control motion sensor 202 to operate at a relatively stable sample rate and perform sample rate conversion. To reduce instances of adjusting the sample rate too quickly and/or too steeply (e.g., when a user switches modes of activities quickly, such as going from standing to running), sample rate controller 212 generates noise having a magnitude equivalent to this sensor noise floor and places the generated noise into the upper frequency bands. As such, motion detection and sensing algorithms may operate on data that can be similar to actual data sampled at a higher sample rate.

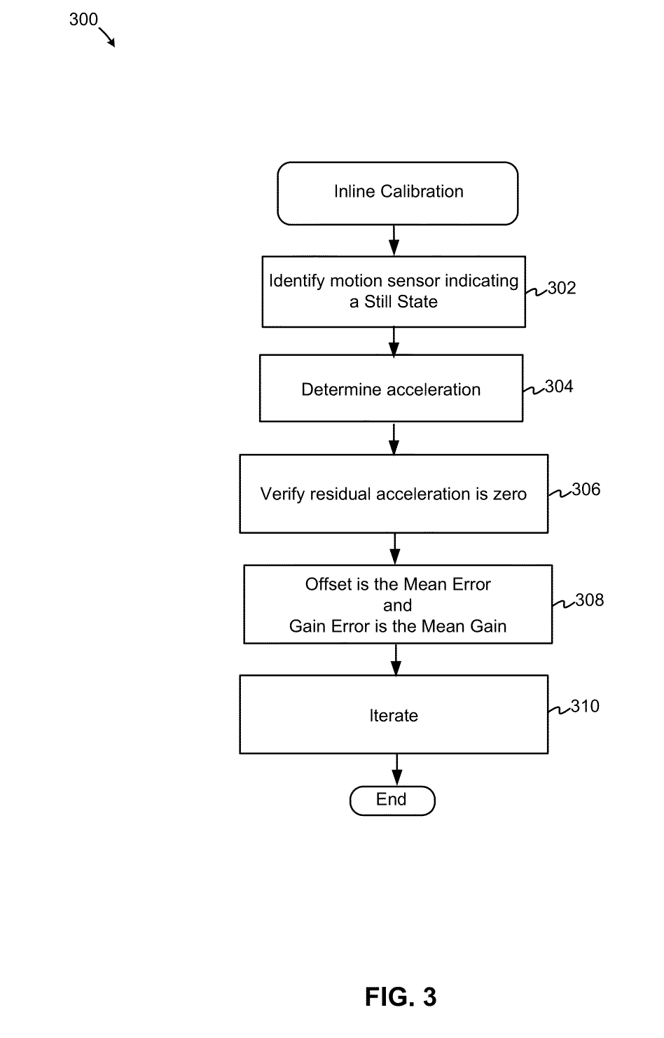

FIG. 3 is an example flow diagram for calibrating a motion sensor in-line, according to some embodiments. At 302, flow 300 identifies whether a motion sensor is indicating that the wearable device is in a “still” state (e.g., with little to no motion). At 304, an acceleration can be determined, for example, due to gravity that is expected to be applied during a present orientation. A determination is made whether a residual acceleration is zero at 306. At 308, an offset is calculated based on a mean error, and a gain error is determined from mean gain. Thereafter, the recalibration process can be iterated to minimize the values of the offset and/or gain error.

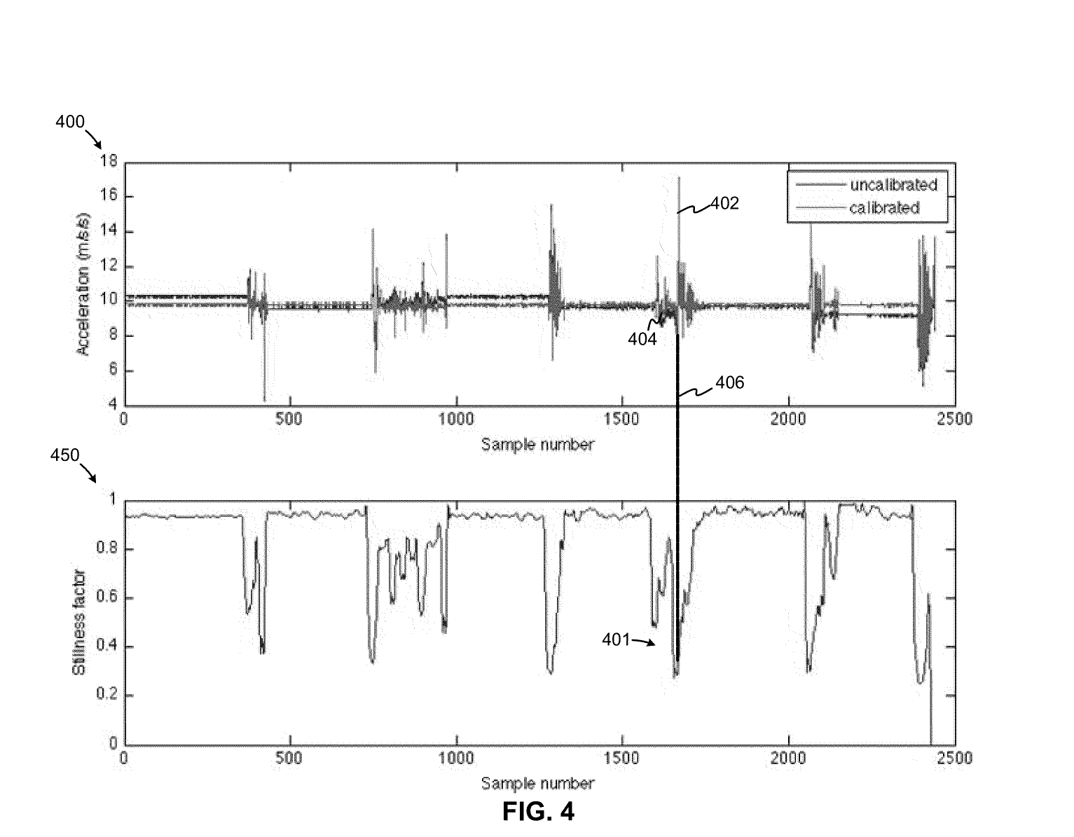

FIG. 4 illustrates a calibrated motion signal, according to at least one example. Diagram 400 depicts a calibrated acceleration signal 402 relative to an uncalibrated acceleration signal 404. As shown in diagram 450, and in view of diagram 400, shows that the calibrated acceleration signal accurately detects changes in a stillness factor 401. In one example, in-line auto-calibration 211 can be configured to calibrate the accelerometer that is providing the calibrated acceleration signal 402.

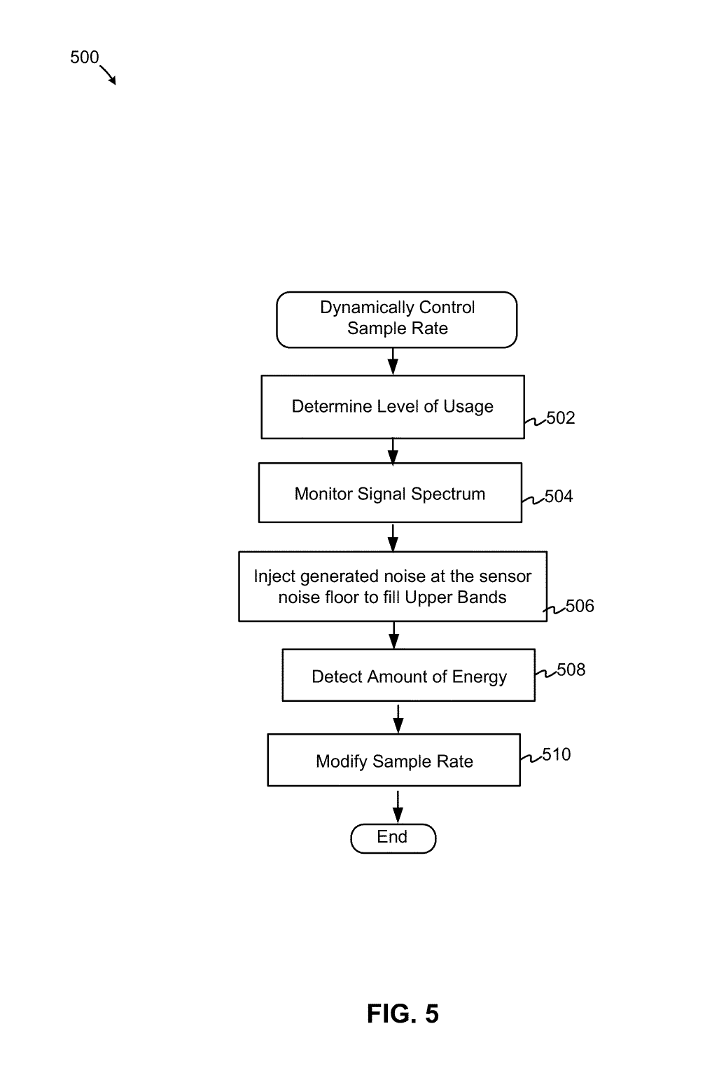

FIG. 5 is an example flow diagram for dynamically controlling a sample rate, according to some embodiments. At 502, flow 500 determines a level of usage based on a level of activity that a user and/or wearable device is experiencing. At 504, flow 500 monitors a spectrum of an accelerometer signal. Generated noise can be injected into the upper bands of frequency, whereby the generated noise has a magnitude equivalent to the sensor noise floor. At 508, an amount of energy is detected relative to the upper frequency bands. If the uppermost bands include energy near the noise floor of the device, then there may be small amounts of information at the corresponding frequencies. If so, the sample rate can be reduced with reduce probabilities of data loss. If there is a relatively large amount of energy in some of the upper bands, there is likely information available at or above the sample rate. Thus, the sample rate can be increased in accordance and/or under the control of sample rate controller 212 of FIG. 2.

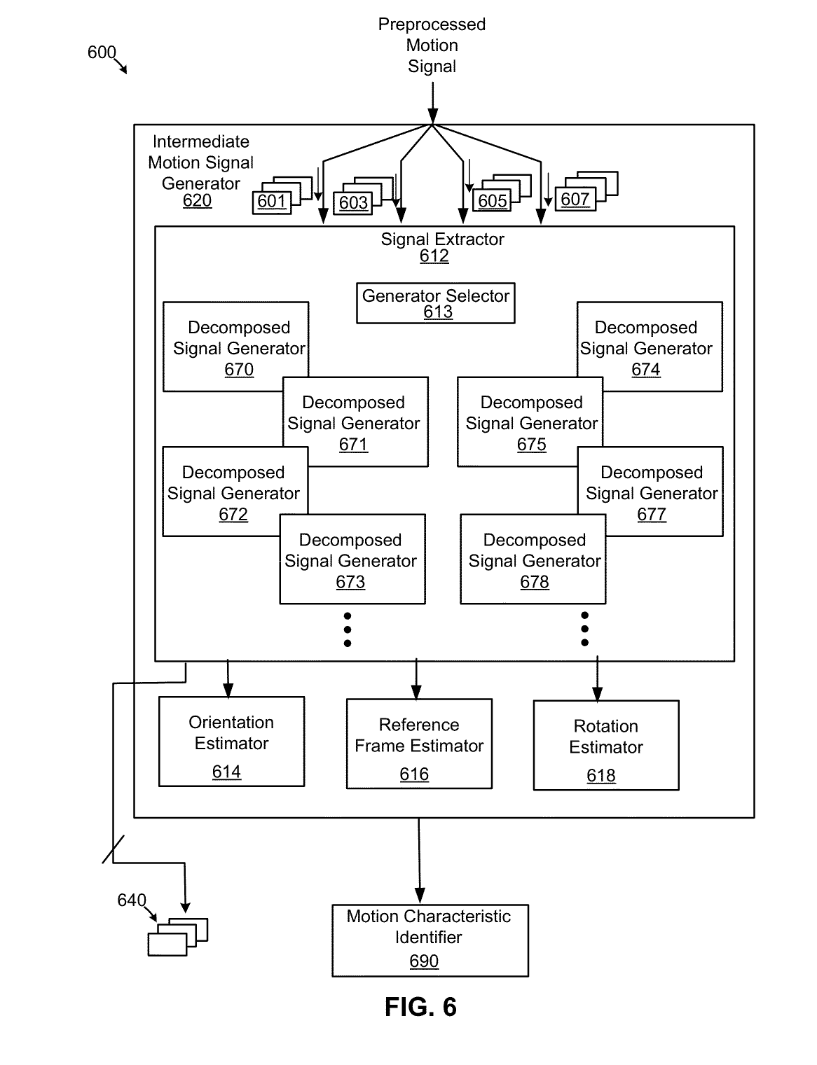

FIG. 6 is an example of an intermediate motion signal generator, according to some embodiments. As shown, intermediate motion signal generator 620 receives preprocessed motion signals, whereby preprocessed accelerometer signals can be viewed as a sum of a number of real-world components, such as an acceleration component 601 due to gravity, one or more applied acceleration components 603 from a frame of reference onto the human body (e.g., a frame of reference can be a floor, a car seat, or any other structure that is either static or in motion), one or more applied acceleration components 605 by the human body onto the wearable device (e.g., from a limb, such as during movement of an arm, etc.), and one or more centripetal acceleration components 607 due to arm rotations or rotations of the frame of reference, such as a car going around a corner. Intermediate motion signal generator 620 is configured to decompose the raw acceleration signal information and thereby deconstruct it into constituent components. For example, intermediate motion signal generator 620 can be configured to separate an accelerometer signal, or other motion-related signals, into constituent components that can be correlated with a phenomena (e.g., velocity, displacement, stillness, etc.) causing or otherwise influencing acceleration rather than, for example, determining acceleration itself. In various embodiments, intermediate motion signal generator 620 can be configured to reconstruct raw accelerated signals from the intermediate motion signals that it generates. Further intermediate motion signal generator 620 can preserve frequencies during the decomposition or signal separation processes.

As shown in FIG. 6, intermediate motion signal generator 620 includes a signal extractor 612, an orientation estimator 614, a reference frame estimator 616, and a rotation estimator 618. Signal extractor 612 is configured to extract intermediate motion signals from the raw acceleration signal. In other words, signal extractor 612 can decompose the raw acceleration or motion signal to form various signals, which can be used to determine an orientation by orientation estimator 614, a reference frame by reference frame estimator 616, and a rotation by rotation estimator 618. Signal extractor 612 includes a number of decomposed signal generators 672 to 677, each of which is configured to generate an intermediate motion signal that can be used by motion characteristic identifier 690 to identify characteristics of the motion (e.g., features). Optionally, signal extractor 612 can include generator selector 613 and can select one or more of decomposed signal generators 672 to 677 to turn one or more of those generators on or off.

Signal extractor 612 can be configured to decompose an accelerometer signal to form the decomposed signals as maximum likelihood estimators, according to some embodiments. Signal extractor 612 can operate according to a presumption that the probability that an orientation in a particular direction can be determined as the maximum likelihood estimation of observing accelerations for a number of possible orientations. That is, signal extractor 612 can operate to set the orientation to be the value of “g” that gives maximum likelihood of P(X|g)*p(g), based on, for example, a Bayesian inference. Further, signal extractor 612 can also presume different estimators are to be viewed as being independent. Thus, signal extractor 612 can form a maximum likelihood estimator of the product of the probability density function, which can be exemplifies as follows:

MLE of P(X|g1)·P(X|g2) . . . .

In some embodiments, intermediate motion signal generator 620 is configured to operate to generate the intermediate motion signals, including stillness. Thus, decomposed signal generator 670 can be configured to determine a “stillness” signal as one of signals 640, for example. As a still device with little to no motion experiences a constant 1 G acceleration, decomposed signal generator 670 can determine stillness by how far away one or more accelerations are from a constant 1 G acceleration. For example, decomposed signal generator 670 can determine the power spectral density over a short sliding window, such as 16 samples. Decomposed signal generator 670 can subtract a value of 1 G from the DC and compute an RMS value of the residual over other frequency bins. Values near zero are deemed as being relatively still (e.g., even if bounded by accelerometer noise). To compute a value of stillness, decomposed signal generator 670 can implement a low-pass filter (e.g., a “better than” a low-pass filter) or an average (e.g., moving average), as higher frequency components can be used to calculate stillness. In some examples, decomposed signal generator 670 can deduce applied accelerations and apply a power spectral density (“PSD”) or wavelet transform. In some other examples, decomposed signal generator 670 can determine whether a distribution of samples match a noise distribution of the accelerometer. Assuming noise is Gaussian with zero-mean and variance equal or substantially equal to the operational characteristics of the accelerometer (or a uniform distribution matching quantization noise), decomposed signal generator 670 can determine a probability that a relatively small number of samples match the distribution and a threshold.

In at least one example, decomposed signal generator 670 can determine a stillness factor over different time periods to provide an indication for how still the device has been recently to detect, for example, sleep versus awake states. First, decomposed signal generator 670 can determine the magnitude of the acceleration, and compute the absolute difference from 1 G. Then, it can form a score such that magnitudes close to 1 G score relatively better than those further away. For example, a score can be calculated as follows: 1/1−abs(ACC_M−1 G). Then, decomposed signal generator 670 can combine the score over multiple samples (e.g., to form the product of the scores for N samples), and vary N to give different lengths of time. Decomposed signal generator 670 can determine the statistics of the product score (e.g. mean, variance, mode, etc.) over different time periods.

Further, decomposed signal generator 670 can determine stillness as an estimator. Consider that the stiller the device, the higher the confidence that an orientation is in the direction of the total acceleration. For a device that is not still, then all directions become more likely. In terms of a probability density function, decomposed signal generator 670 can model p(X|g) as a Gaussian distribution of theta and phi, with mean equal to X and standard deviation a function of the stillness (e.g., the less still, the wider larger the standard deviation). So the probability of seeing X given g is approximately the Gaussian of (|X-g|/sigma) where sigma is around 1/stillness, or:

P(X|g)·Erf(|X−g|/(1/stillness)

Decomposed signal generator 671 is configured to form a decomposed signal component, such as an applied force. Consider that the closer an applied force is to 1 G, the more confidence there is that an orientation is the norm of the applied force. Decomposed signal generator 671 can presume that applied forces follow an activation function in size (i.e., larger forces are less likely according to a 1/f rule), which can be viewed as being equivalent to an exponential distribution. Note that this can be a maximum entropy assumption (i.e., an example of a minimum assumption). Thus, the PDF can be approximated as follows:

P(X|g)˜e(−1·|X−g|)

In some cases, the applied acceleration can be relative to the device (excluding gravity). For example, if a user moves an arm back and forth, that person applies an acceleration that is in a consistent direction relative to the device irrespective of how the user's arm is oriented. Further, the applied acceleration can be relative to the world (excluding gravity). For example, if a user jumps up and down, that person applies a vertical (in world coordinates) acceleration to the device for the period of time when that person's feet are driving off the ground. Note that clapping will show applied accelerations that are not vertical in world coordinates.

Decomposed signal generator 672 is configured to form a decomposed signal component, such as a continuity estimator. Consider that an orientation matching parameters to a previous orientation is more likely than there being a relatively large difference between the orientation separated by time. Decomposed signal generator 672 can use an activation function for the size of orientation changes.

Thus:

P(g|g−1)˜e(−|g−g1|̂2/2·sigma&2)

Decomposed signal generator 673 is configured to generate a decomposed signal component, such as vertical acceleration. Consider that is generally difficult to sustain acceleration that is not parallel to the ground for an extended period (e.g., other than rocket ships, missiles, or planes nose-diving into the ground). Accelerations perpendicular to the ground and an in upward direction that lead to extensions of greater than a meter or so (e.g., 1 g for 0.5 seconds or so) lead to a loss of contact with the ground and the inability to provide a further acceleration. Thus, accelerations towards the ground that persist for more than a few 100 ms or meters are typically free-fall (and hence oriented directly to the ground) or lead to dangerous impacts that are likely rare. It will be seen that an orientation error leads to a dc acceleration that might imply take-off or crash. Given a previously determined vertical acceleration, the PDF is as follows:

P(X|g)˜1/((THRESHOLD−sum(acceleration over last k samples)·Z-AXIS−(X−g)·g)

Decomposed signal generator 674 is configured to generate a decomposed signal component, such as a minimum energy constraint. Decomposed signal generator 674 can be configured operate on an assumption that a human is an efficient system and uses a minimum amount of minimum energy to achieve a particular goal. The energy used can be set as the sum over suitable samples of the “acceleration·distance”. Provided that relevant masses are deemed constant over this period, an exponential distribution can provide an estimator as follows:

P(X|g)˜e−((1+(X−g)·(v*t+0.5*(X−g)·*t*t))

Decomposed signal generator 675 is configured to generate a decomposed signal component, such as a minimum velocity. Decomposed signal generator 675 can assume that a human generates minimum velocity to achieve a given task. This is particularly useful as orientation errors lead to rapidly rising calculated velocities. Using an activation function:

P(X|g)˜e−(v+(X−g)t)

Decomposed signal generator 677 is configured to generate a decomposed signal component, such as curvature. Decomposed signal generator 677 is configured to assume that predominant orientation changes are a result of a device following an arc of non-zero radius about an axis perpendicular to gravity. Decomposed signal generator 677 is further configured to estimate curvature as a “cross product” of the normalized (i.e., unit) velocity with a delayed version of the same. The magnitude of this cross product is sine of the angle subtended, and the direction is the axis of rotation. Thus, decomposed signal generator 677 is configured to can rotate this axis from a device coordinate system to a world coordinate system using a previous orientation to provide a rotation about an axis perpendicular to gravity.

Decomposed signal generator 678 is configured to generate a decomposed signal component, such as a correlated signal. For example, decomposed signal generator 678 can assume that acceleration due to gravity is poorly or weakly correlated with an applied acceleration. So a PDF can be used to determine minimal correlation between gravity and the applied force.

Based on or more of the foregoing, orientation estimator 614 can use the decomposed signals to determine an orientation. Orientation estimator 614 can determine an orientation based on a combination of the PDFs into a PDF, for example, by multiplication. Then, the maximum likelihood estimator is as follows:

L˜Sum ln(P(X|g)

Orientation estimator 614 can maximize this estimator for two possible angles for g (theta, phi), and can use the previous orientation as a starting point, s. Thus, orientation estimator 614 can determine an estimate for the orientation, g.

In summary, orientation can be determined based on one or more of: a previous orientation is close to the current one (when wearable device is still), a direction of the total acceleration, which is likely to be close to the direction of gravity, when a device has an acceleration whose magnitude is close to 1 G, a probability that sustained accelerations perpendicular to the ground is low, a probability that a wearable device is at a high velocity is low, minimum energy trajectories are preferred, and an orientation does not change without rotation, thus, centripetal accelerations arise.

Signal extractor 612 can also include other decomposition signal generators that are not shown. For example, a decomposition signal generator can establish an applied acceleration, such as:

X−g

A decomposition signal generator can establish a world-applied acceleration by rotating the applied acceleration using, for example, Quaternions by the orientation. A decomposition signal generator can establish a velocity and displacement (e.g., in the device and world coordinates) by using the integrals of the acceleration. Stillness can be used to reset velocity and displacement to prevent issues. A decomposition signal generator can establish a centripetal acceleration. A decomposition signal generator can establish a linear acceleration, which can be derived from the applied accelerations minus centripetal acceleration. A decomposition signal generator can establish a radius and direction of curvature from centripetal acceleration (e.g., a cross-product of velocity and acceleration to determine an axis of rotation and angular velocity in rad/sec). A decomposition signal generator can establish a cross-correlations between signals as it can be useful to examine cross-correlations between some of the signals, whereby additional signals may be determined by cross-correlation. Such signals can be output as signals 640 for use by another component of the various embodiments.

Reference frame estimator 616 is configured to estimate a frame reference and associated information, such as a moving car or a chair providing a static force. Rotation estimator 618 is configured to estimate rotation between coordinate systems, and can operate similarly to decomposed signal generator 677. Outputs of intermediate motion signal generator 620 are transmitted to motion characteristic identifier 690.

According to some examples, intermediate motion signal generator 620 is configured to operate based on probabilities that: smaller applied forces are more likely than larger ones, smaller velocities are more likely than larger ones, energy is likely to be approximately minimized, orientation changes are more likely when the angular velocity is larger, the wearer is likely to be within a few meters of the ground, orientation changes are approximately independent of applied forces excluding centripetal forces, the fact that something is moving back and forth does not mean that an orientation is changing back and forth, frame of reference forces are generally closer to the perfectly vertical or perfectly horizontal, rotations with a radius of curvature larger than human joints are likely to be caused by rotations of the frame of reference, although this is not a closer (momentum-conserving) system, smaller changes in momentum (angular plus linear) are more likely than large ones, slower orientation changes are more likely than rapid ones, and the like.

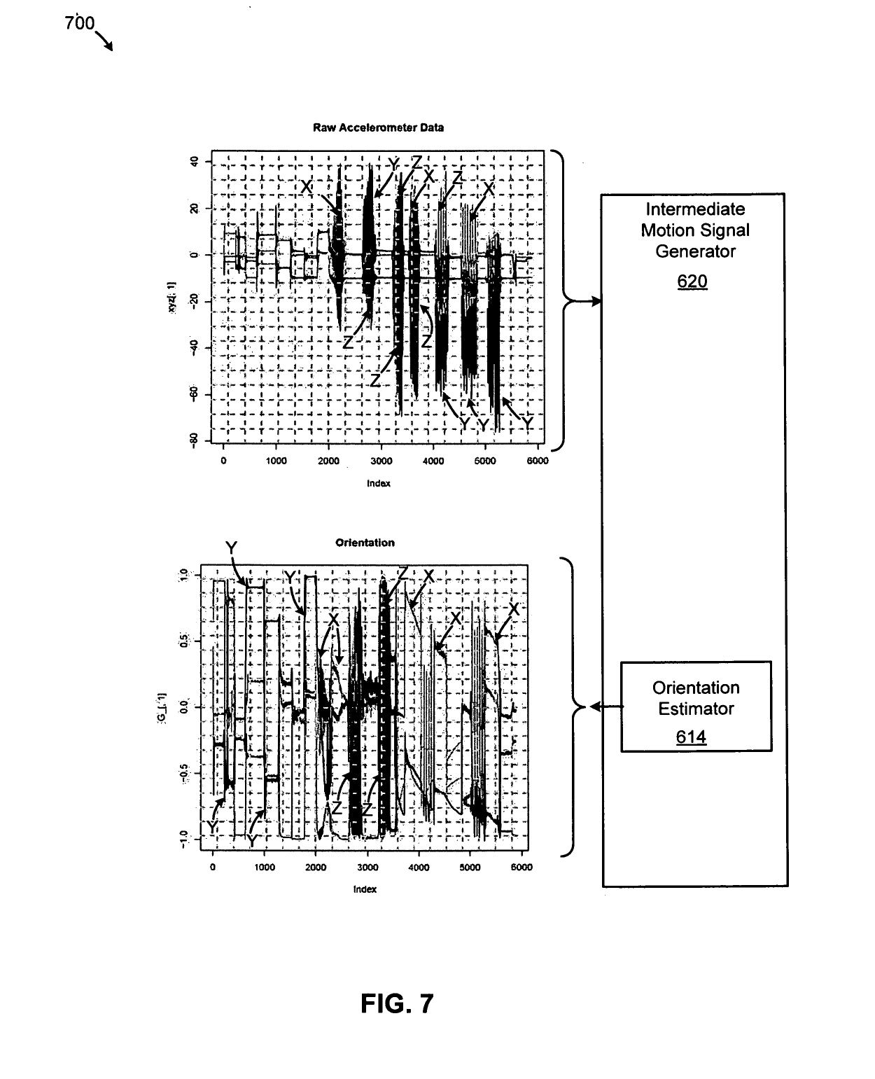

FIG. 7 is a diagram depicting an estimated orientation derived from an intermediate motion signal generator, according to some embodiments. Diagram 700 shows intermediate motion signal generator 620 receiving accelerometer data and orientation estimator 614 generating a corresponding orientation. Diagram 700 is merely but an example to depict the functionalities of intermediate motion signal generator 620; FIG. 7 is not intended to be limiting.

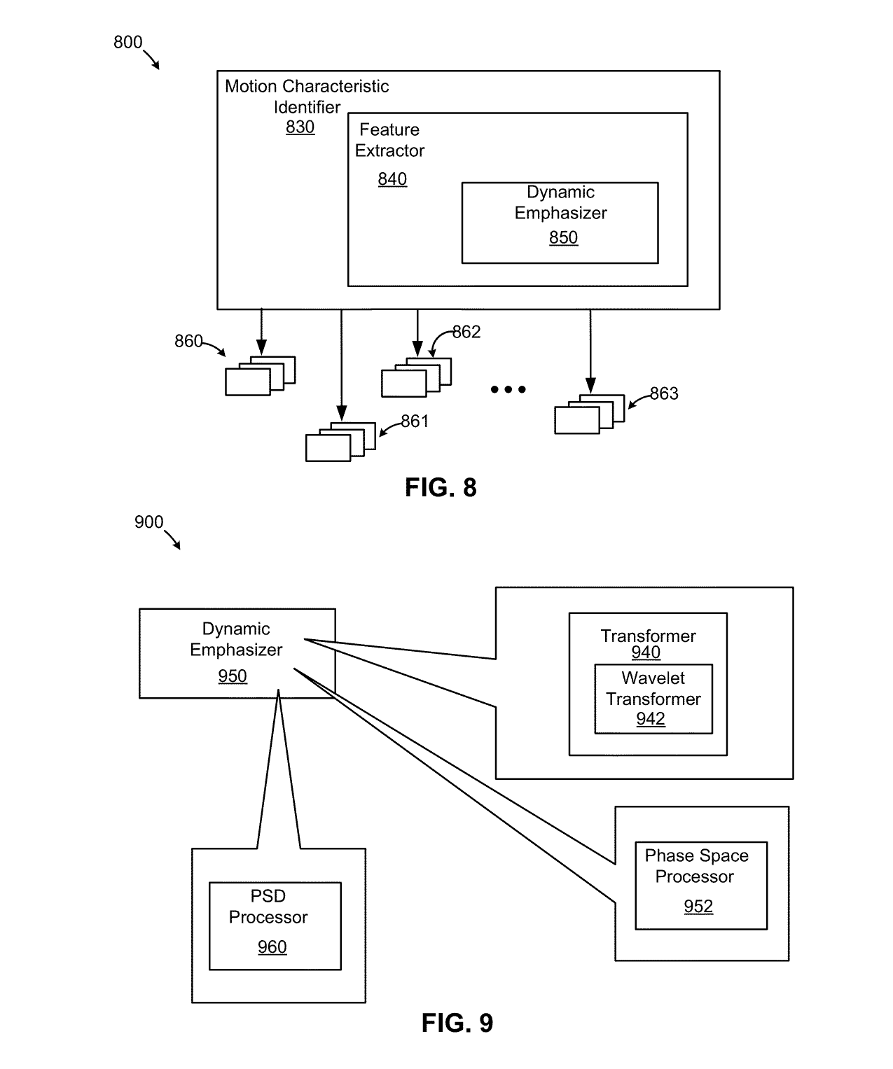

FIG. 8 is a diagram depicting a motion characteristic identifier, according to some examples. Motion characteristic identifier 830 is configured to analyze the decomposed signals and other information from intermediate motion signal generator 620 of FIG. 6 to identify certain attributes of motion based on the decomposed signals. As shown, motion characteristic identifier 830 includes a feature extractor 840 which, in turn, includes a dynamic emphasizer 850. Feature extractor 840 is configured to extract the features that are identifiable from the decomposed signals of a motion and to generate feature data 860 to 863. In particular, feature extractor 840 identifies and extracts the features based on the functionality of dynamic emphasizer 850 which is configured to identify transients variability in motion related signals and emphasize the dynamism of such signals.

In some embodiments, feature extractor 840 is configured to turn signals into a number of parameters that can be used to drive a classifier. Such features can be a particular type of summary of the signal, whereby the features can be compact (e.g., the amount of information provided is minimized), relevant (e.g., the information provided is that information that is most closely aligned with the activities being detected), of a suitable spatial-temporal resolution (e.g., features that have a 1 Hz resolution may not be useful for detecting activities that are of short durations, such as 100 ms, and independent, and efficient computationally.

FIG. 9 is an example of a dynamic emphasizer 950, according to some embodiments. As shown, dynamic emphasizer 950 can be a transformer 940, which can operate provide any type of transform whether in the time or frequency domain or otherwise. In some embodiments, transformer 940 is a wavelet transformer 942. Wavelet transforms can be produced by successively downsampling a signal by a power of 2, and convolving a kernel with each generated downsampled signal. The kernel can be designed to emphasize dynamics (i.e., transients) in such a way that the output of the wavelet transform at each sample rate is independent of the output at other sample rates. That is, the kernel emphasize can, for each sample rate, dynamics that are of that temporal scale. Methods exist to perform wavelet transforms efficiently (order N, rather than order N log N as for Fourier transforms). A wavelet can be viewed as separating the signal—at every level—to expose the “details” and “averages” and then decomposing the “averages” into more detail at a lower temporal scale, and so on. Wavelet transformer 942 can provide a good independence between features, can have relatively high temporal resolution for fast transients and dynamics, can have relatively low temporal resolution for slow transients that do not need any higher resolution, and is computationally efficient. Wavelet transforms can have good noise-rejection properties with relatively little smoothing of the signal. Since the signal is decomposed into sets of “detail” at different temporal resolutions, irrelevant (i.e., subthreshold) details can be rejected without loss of relevant high-resolution detail. Wavelets can be typically short filters over only a few coefficients that are applied continuously to the sub-sampled signal. In other embodiments, dynamic emphasizer 950 can be implemented as a phase space processor 952. In particular, phase space processor 952 can be configured to perform moments of the phase space, and can be generated by taking the phase space of the signals and then transforming them using wavelet transforms and other techniques such as power spectral density and window moving averages. Moments of the phase space (i.e. sum over k (acc/\N*y−̂N)−sum over k (acc*y) where y is the integral or differential of the acceleration where k is a number of samples that may be varied. Also shown in FIG. 9, dynamic emphasizer 950 can also include a PSD processor 960 can be configured to implement power spectral density functionality among others. For example, while moving averages and power spectral densities may be used in the various implementations, wavelet transformer 942 facilitates effective and efficient motion and activity determinations.

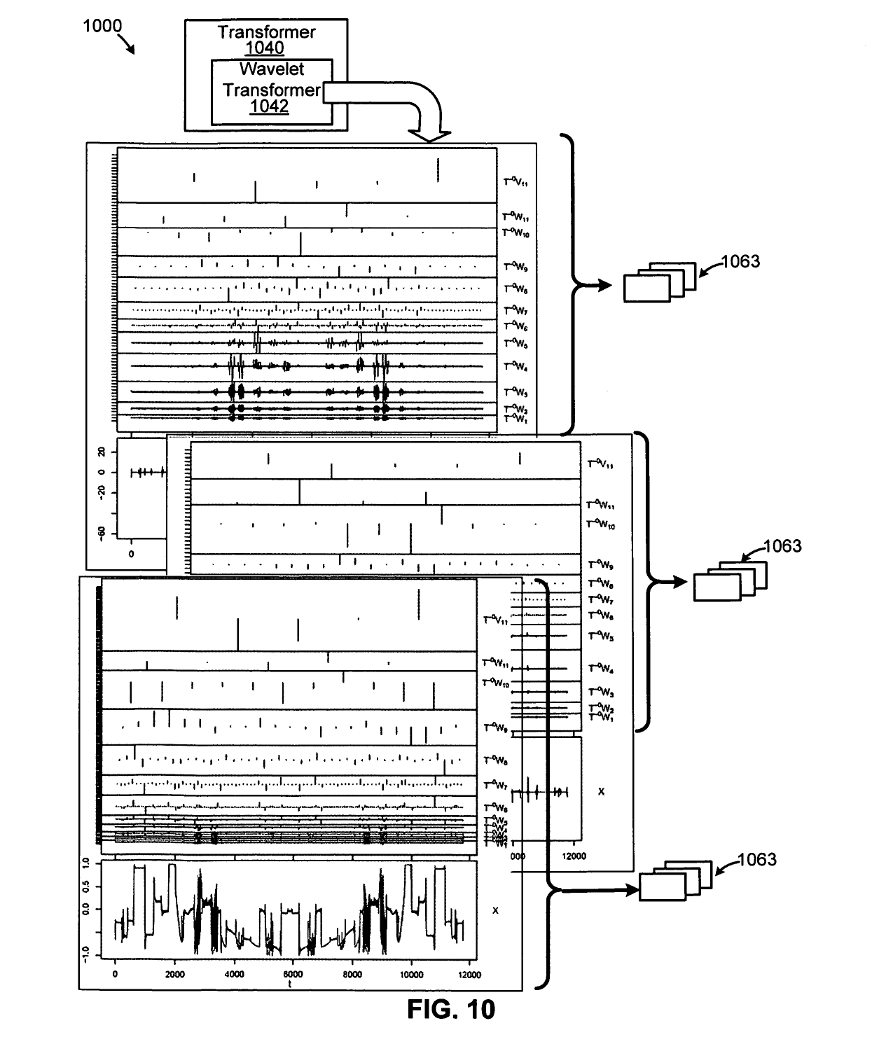

FIG. 10 depicts extracted features according to some embodiments. As shown, diagram 1000 includes transformer 1040, which in turn, includes wavelet transformer 1042. Wavelet transformer 10,042 is configured to generate feature data 1063.

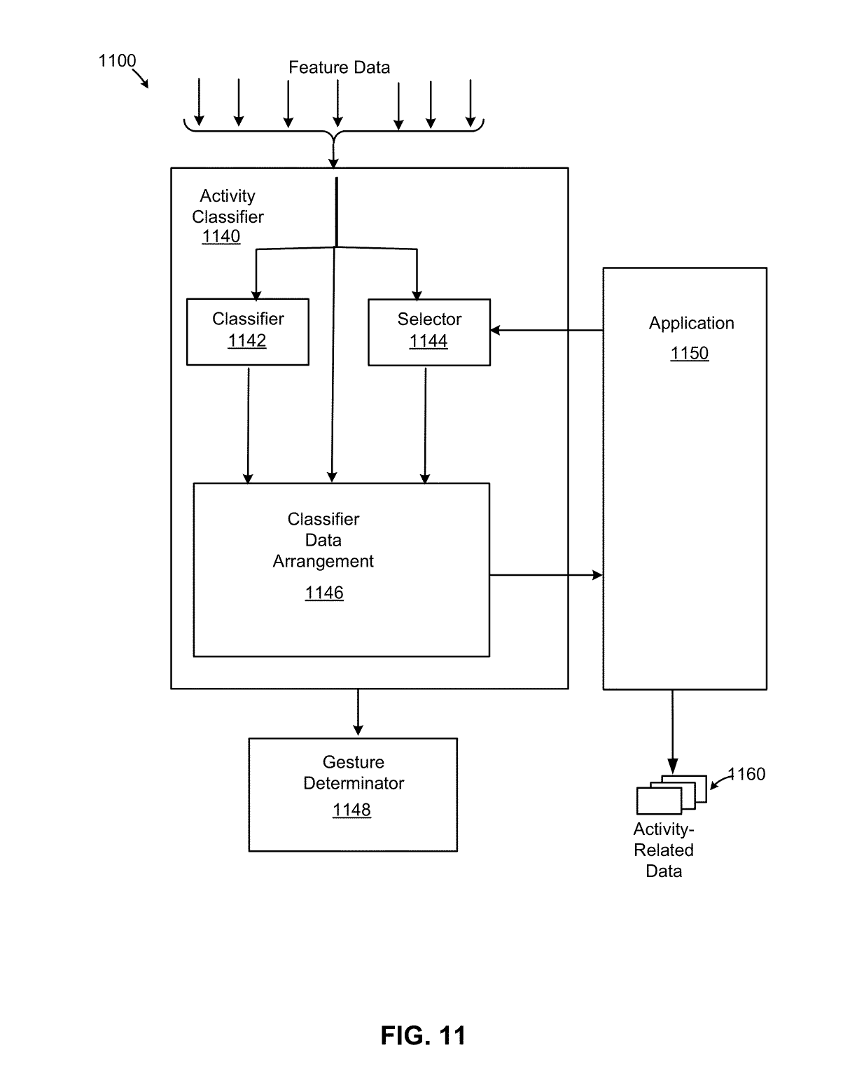

FIG. 11 depicts an activity classifier, according to some embodiments. Activity classifier 1140 includes a classifier 1142 in a selector 1144, as well as a classifier data arrangement 1146. In application 1150 such as a sleep management or pedometer application, is configured to exchange information with activity classifier 1140. Classifier data arrangement 1146 is an arrangement of data including various feature data set, and can be a matrix of data. The feature data represents reduced data spaces that can be compared against the data in classifier data arrangement 1146 to determine matches and to identify portions of activity in activities itself. Selector loan 40 is configured to select the subset of the features that are of interest to the application. For example, sleep management applications are interested in feature that relate to stillness and other characteristics of sleep. In various embodiments, activity classifier includes a classification parametric modeling system. In one example, activity classifier implements a Markov modeling and aggregation system. Classifier 1142 and/or classifier data arrangement 1146 can include a number (e.g., anywhere from a few to hundreds or more) of, for example, YES or NO questions to which the aggregation of the responses are used to classify and/or identify micro-activities and portions of activities that correspond to gestures or portions of motion.

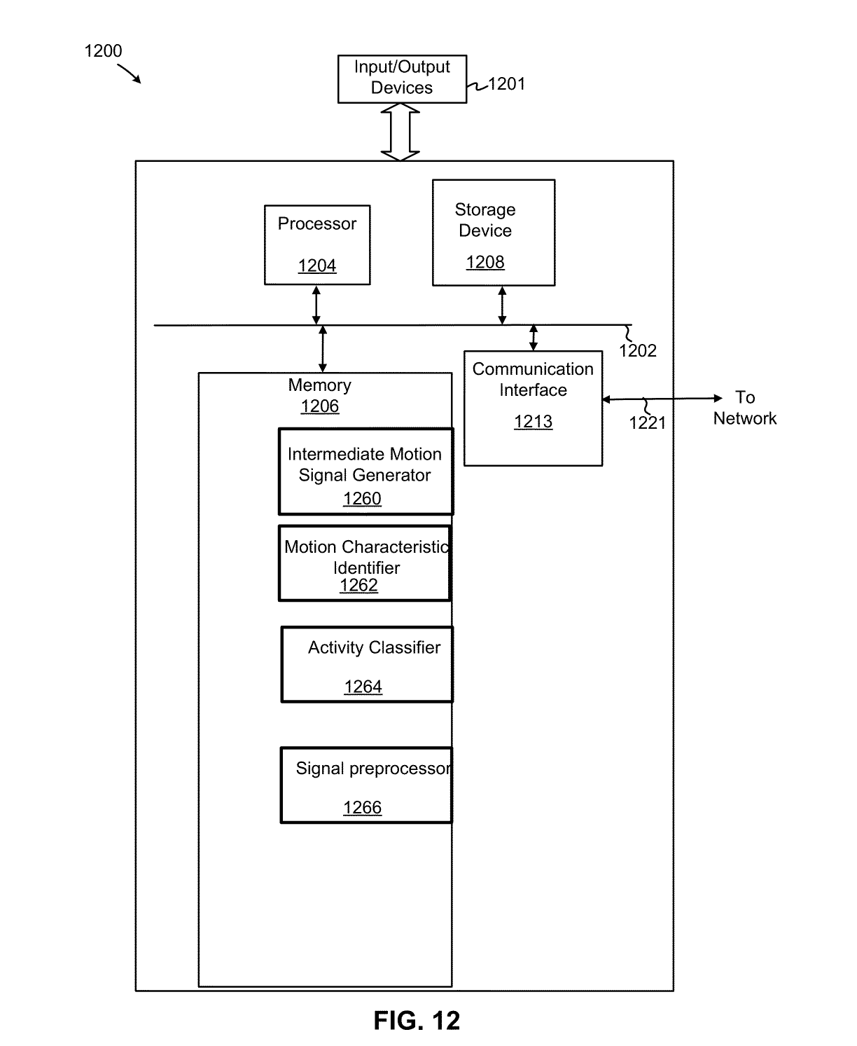

FIG. 12 illustrates an exemplary computing platform disposed in a wearable device or otherwise implements at least some of the various components in accordance with various embodiments. In some examples, computing platform 1200 may be used to implement computer programs, applications, methods, processes, algorithms, or other software to perform the above-described techniques.

In some cases, computing platform can be disposed in an ear-related device/implement, a mobile computing device, or any other device.

Computing platform 1200 includes a bus 1202 or other communication mechanism for communicating information, which interconnects subsystems and devices, such as processor 1204, system memory 1206 (e.g., RAM, etc.), storage device 12012 (e.g., ROM, etc.), a communication interface 1213 (e.g., an Ethernet or wireless controller, a Bluetooth controller, etc.) to facilitate communications via a port on communication link 1221 to communicate, for example, with a computing device, including mobile computing and/or communication devices with processors. Processor 1204 can be implemented with one or more central processing units (“CPUs”), such as those manufactured by Intel® Corporation, or one or more virtual processors, as well as any combination of CPUs and virtual processors. Computing platform 1200 exchanges data representing inputs and outputs via input-and-output devices 1201, including, but not limited to, keyboards, mice, audio inputs (e.g., speech-to-text devices), user interfaces, displays, monitors, cursors, touch-sensitive displays, LCD or LED displays, and other I/O-related devices.

According to some examples, computing platform 1200 performs specific operations by processor 1204 executing one or more sequences of one or more instructions stored in system memory 1206, and computing platform 1200 can be implemented in a client-server arrangement, peer-to-peer arrangement, or as any mobile computing device, including smart phones and the like. Such instructions or data may be read into system memory 1206 from another computer readable medium, such as storage device 1208. In some examples, hard-wired circuitry may be used in place of or in combination with software instructions for implementation. Instructions may be embedded in software or firmware. The term “computer readable medium” refers to any tangible medium that participates in providing instructions to processor 1204 for execution. Such a medium may take many forms, including but not limited to, non-volatile media and volatile media. Non-volatile media includes, for example, optical or magnetic disks and the like. Volatile media includes dynamic memory, such as system memory 1206.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 1202 for transmitting a computer data signal.

In some examples, execution of the sequences of instructions may be performed by computing platform 1200. According to some examples, computing platform 1200 can be coupled by communication link 1221 (e.g., a wired network, such as LAN, PSTN, or any wireless network) to any other processor to perform the sequence of instructions in coordination with (or asynchronous to) one another. Computing platform 1200 may transmit and receive messages, data, and instructions, including program code (e.g., application code) through communication link 1221 and communication interface 1213. Received program code may be executed by processor 1204 as it is received, and/or stored in memory 1206 or other non-volatile storage for later execution.

In the example shown, system memory 1206 can include various modules that include executable instructions to implement functionalities described herein. In the example shown, system memory 1206 includes a signal preprocessor 1266, an intermediate motion signal generator 1260, a motion characteristic identifier 1262, and an activity classifier 1264, which can be configured to provide or consume outputs from one or more functions described herein.

In at least some examples, the structures and/or functions of any of the above-described features can be implemented in software, hardware, firmware, circuitry, or a combination thereof. Note that the structures and constituent elements above, as well as their functionality, may be aggregated with one or more other structures or elements. Alternatively, the elements and their functionality may be subdivided into constituent sub-elements, if any. As software, the above-described techniques may be implemented using various types of programming or formatting languages, frameworks, syntax, applications, protocols, objects, or techniques. As hardware and/or firmware, the above-described techniques may be implemented using various types of programming or integrated circuit design languages, including hardware description languages, such as any register transfer language (“RTL”) configured to design field-programmable gate arrays (“FPGAs”), application-specific integrated circuits (“ASICs”), or any other type of integrated circuit. According to some embodiments, the term “module” can refer, for example, to an algorithm or a portion thereof, and/or logic implemented in either hardware circuitry or software, or a combination thereof. These can be varied and are not limited to the examples or descriptions provided.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

1. A method comprising:
receiving data representing a motion sensor signal from a motion sensor disposed in a wearable device;
generating a plurality of intermediate motion signals from the motion sensor signal;
identifying characteristics of motion based on the intermediate motion signals to form motion characteristics data; and
determining an activity based the motion characteristics data.

2. The method of claim 1, wherein receiving the data representing the motion sensor signal from the motion sensor further comprises:
receiving accelerometer data representing an acceleration signal from an accelerometer.

3. The method of claim 1, wherein identifying the characteristics of motion comprises:
extracting features of the intermediate motion signals to form the motion characteristics data.

4. The method of claim 1, wherein identifying the characteristics of motion comprises:
transforming the intermediate motion signals to form the motion characteristics data.

5. The method of claim 4, wherein transforming the intermediate motion signals comprises:
transforming the intermediate motion signals to determine features,
wherein the features differ in terms of temporal variability.

6. The method of claim 1, wherein generating the plurality of the intermediate motion signals comprises:
decomposing the motions sensor signal to form one or more decomposed signals.

7. The method of claim 6, wherein decomposing the motions sensor signal to form the one or more decomposed signals comprises:
forming signals representing one or more of an orientation, an applied acceleration, and a centripetal acceleration.

8. The method of claim 7, further comprising:
extracting features from the signals representing one or more of the orientation, the applied acceleration, and the centripetal acceleration.

9. The method of claim 8, wherein extracting features from the signals comprises:
performing a wavelet transformation on one or more signals from the signals representing one or more of the orientation, the applied acceleration, and the centripetal acceleration.

10. The method of claim 8, wherein extracting features from the signals comprises:
identifying representations of the wavelet transformation of at least one signal at different sample rates.

11. The method of claim 10, wherein identifying representations of the wavelet transformation comprises:
identifying representations of the wavelet transformation produced by successively downsampling the at least one signal.

12. The method of claim 1, further comprising:
combining the plurality of intermediate motion signals.

13. The method of claim 12, wherein combining the plurality of intermediate motion signals comprises:
generating one or more decomposed signal components using one or more estimators; and
forming a product of a plurality of probability density functions (“PDFs”) for the one or more decomposed signal components.

14. The method of claim 13, further comprising:
performing a wavelet transformation on at least one decomposed signal component.

15. The method of claim 14, wherein performing the wavelet transformation comprises:
downsampling the at least one decomposed signal component; and
performing the wavelet transformation to form a plurality of extracted features.

16. An apparatus comprising:
a wearable housing;
a motion sensor configured to sense motion associated with the wearable housing and to generate a motion sensor signal;
an intermediate motion signal generator configured to receive the motion sensor signal, and further configured to generate intermediate motion signals;
a motion characteristic identifier configured to identify characteristics of motion based on the intermediate motion signals to form motion characteristics data; and
an activity processor configured to identify an activity based on the motion characteristics data.

17. The apparatus of claim 16, wherein the motion characteristic identifier comprises:
a feature extractor configured to extract features of the intermediate motion signals to form the motion characteristics data.

18. The apparatus of claim 16, wherein the feature extractor further comprises:
a transformer configure to identify temporal variability.

19. The apparatus of claim 18, wherein the transformer is configured to transform extracted features in terms of the temporal variability.

20. The apparatus of claim 16, wherein the motion characteristic identifier comprises:
a wavelet transformer.
